(19) United States
(12) Patent Application Publication
(10) Pub. No.: US 2019/0227053 A1
(43) Pub. Date: Jul. 25, 2019

(54) BIO-ELECTRIC NOSE

(71) Applicants: NEW YORK UNIVERSITY, New York, NY (US); NORTHWESTERN UNIVERSITY, Evanston, IL (US)

(72) Inventors: Dmitry RINBERG, New York, NY (US); Erez SHOR, New York, NY (US); Thomas BOZZA, New York, NY (US)

(21) Appl. No.: 16/312,973

(22) PCT Filed: Jun. 23, 2017
(86) PCT No.: PCT/US2017/039066

§ 371 Date: Dec. 21, 2018

Related U.S. Application Data
(60) Provisional application No. 62/354,627, filed on Jun. 24, 2016.
(60) Provisional application No. 62/354,629, filed on Jun. 24, 2016.

(51) Int. Cl. G01N 33/50; G01N 33/00

(52) U.S. Cl. CPC G01N 33/5088; A01K 2227/10; G01N 33/0001

(57) ABSTRACT

An animal-based chemical detectors that can be customized to detect a wide variety of chemicals under real-world conditions. Methods and systems for brain/machine interface devices using electrodes to read odor-signatures from the patterns of activated glomeruli, for example in the rodent olfactory bulb.

### TECHNICAL FIELD

The present disclosure relates generally to systems and methods for ligand detection.

### BACKGROUND

Chemical detection is a critical capability for a wide range of efforts promoting national security and human health. When it comes to sensitivity, flexibility and specificity, no artificial chemical sensor outperforms the nose. This is particularly true in unpredictable, rapidly changing environments—conditions under which biological systems excel. This is why humans rely on the olfactory abilities of dogs and other animals to track and pursue missing people, locate explosives and drugs in airports, and even to diagnose latent infections and illnesses such as cancer and tuberculosis. Despite progress in developing artificial gaseous chemical sensors, biological systems still substantially outperform artificial systems in their sensitivity, dynamic range and versatility. Consequently, the use of biological olfactory systems to perform real-world tasks is constantly expanding into many new domains.

While animals are used frequently to perform chemical recognition, a major disadvantage of biological noses are the difficulty in getting animals to report the detection of a desired chemical and the inability to tune the nose towards the sensitive detection of specific compounds. Getting animals to report chemical detection often requires extensive training—for example, getting dogs to recognize a specific chemical and sit upon identifying this stimulus. While the olfactory system is quite reliable under varying conditions, an animal's behavior can vary dramatically depending on surrounding conditions. Furthermore, training animals to report different odors or different concentrations is virtually impossible.

The olfactory system is a highly sensitive and flexible chemical detection system. Odorous chemicals in the environment are inhaled and evoke unique patterns of activity across an array of structures called glomeruli which are located in the olfactory bulb (the first part of the brain that receives olfactory information). There are over 2000 glomeruli in the rodent olfactory bulb, and the presence of even simple chemical stimuli activate many glomeruli in broad overlapping patterns creating an odor signature.

### SUMMARY

One embodiment relates to a chemical sensor comprising: an electrode assembly; a chemical detector in communication with the electrode assembly, wherein the chemical detector comprises a glomerulus.

One embodiment relates to a method of detecting the concentration of a present chemical comprising: binding a chemical to an olfactory receptor, located in an olfactory sensory neuron in the nasal epithelium and projecting its axon to the olfactory bulb (for example, an olfactory bulb of a normal or genetically manipulated non-human mammal); detecting an electrochemical change in the olfactory bulb; and identifying the concentration of the chemical based upon the detected electrochemical change.

One embodiment relates to a method of detecting the concentration of a mixture of chemicals in different concentration comprising: binding a chemical to a olfactory neuron; detecting an electrochemical change in the olfactory bulb; and identifying the compounds and concentration of the chemical based upon the detected electrochemical change.

One embodiment relates to a method for distributed odor mapping of areas comprising: providing a recording system, wireless communications, GPS and a chemical sensor in communication with each other; detecting odors by freely moving organisms and recording information regarding positions of the organisms; and compiling an odor map of a target area given the recorded information.

One embodiment relates to a system for driving a motorized vehicle based on odor detection comprising; binding a motorized vehicle, control system, odor and concentration classifier and the said chemical sensor; driving the motorized vehicle to odor sources based on algorithm processing odor and concentration gradients detected by the chemical sensor.

One embodiment relates to a system for a non-invasive diagnosis of diseases, where a disease marker is chemically detected by the system in a sample of breathed air, tissue, bodily fluid, feces, urine or sweat.

One embodiment relates to a transgenic non-human mammal having a gene sequence encoding for the targeted or modified expression of one or more olfactory receptor genes so that they wire to defined regions of the olfactory bulb to optimize use in a brain machine interface.

One embodiment relates to a method for creating a chemical sensor comprising: modifying a genetic sequence of an organism; directing expression of olfactory receptors with specificity for a selected chemical; generating ectopic or multiple dorsal glomeruli having specificity for the selected chemical; and placing an electrode array adjacent to the dorsal glomeruli.

One embodiment relates to a method for, and system containing, mutating a giving odorant receptor to increase affinity for a given chemical of interest for inclusion into a chemical sensor (bio-electric nose).

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter appearing at the end of this disclosure are contemplated as being part of the subject matter disclosed herein.

### BRIEF DESCRIPTION OF DRAWINGS

The foregoing and other features of the present disclosure will become more fully apparent from the following description and appended claims, taken in conjunction with the accompanying drawings. Understanding that these drawings depict only several implementations in accordance with the disclosure and are therefore, not to be considered limiting of its scope, the disclosure will be described with additional specificity and detail through use of the accompanying drawings.

FIG. 1 illustrates sample odor maps (20 um spacing electrode).

FIG. 2 illustrates classification accuracy for two tested electrodes and seven odors presented.

FIG. 3A shows setup of calibrated odor presentation system for bio-electronic nose; FIG. 3B shows preparation of Bio-electronic interface to animal (implantation of custom-made electrodes on the olfactory bulb and pressure sensor);

FIG. 3C shows setup and factory calibration of bio-electronic nose by presentation of multiple odors and processing of pressure and electrode outputs; FIG. 3D shows Utilization, where the calibrated bio-electronic nose can detect and classify odors in real-world environments.

FIG. 4A: In vivo calcium imaging of odor evoked activity in olfactory bulb glomeruli. Confocal image shows dorsal glomeruli (green). Responses to two different odorants are shown. Warm colors represent greater increases in fluorescence. FIG. 4B: Schematic of transgenic strategy to target olfactory receptors for dorsal expression. For illustration, the example shows various receptors (ORs 1-5) that are genetically targeted for expression using a dorsal receptor gene. The resulting expression of the exogenous receptors produces glomeruli (right images) that are in a similar dorsal position as the endogenous receptor (left image).

FIG. 5A illustrates a surface probe array for cortical recordings with an 8×8 electrode array and 55 um spacing; FIG. 5B illustrates a surface probe array for cortical recordings with an 12×10 electrode array and 20 um spacing.

FIG. 6A: Local Field Potential signal in the dorsal OB has an odor dependent signature, as seen upon the presentation of odor 2-hexanone. Top: Activity recorded by the surface electrode located in the posterior part of the olfactory bulb. Middle: Spectrogram of the LFP, showing distinctive activity in response to 2-hexanone. White trace indicated the state of the odor presence (high is odor presented). Bottom: respiratory activity. B. Interpolated map of the LFP power distribution for two different odors (top: 2-hexanone, bottom: 4-methoxylacetophenone) recorded with the probe placed on the thinned skull. Gray circles are electrode positions.

FIG. 7: Experimental setup. The anesthetized mouse is positioned in head fixation setup. A sniffing cannula for pressure measurement is implanted in one of the nostrils and connected to the pressure transducer. A craniotomy is done above one of the OBs and an electrode array is positioned on the surface of the bulb. The array is connected to the 64/128 preamplifiers and multiplexing system, which output is sent to the A/D board (not shown). During the experimental session, a final valve, which delivers odors, is triggered at the onset of exhalation, measured by the pressure transducer (A, time point 1). A typical pressure waveform is shown on the top left panel (A). Such triggering ensures that when a mouse starts inhaling an odor (time point 3), the odor concentration has reached a steady state (time point 2). The odor delivery system is calibrated prior every experimental session using photoionization detector (PID). The typical profiles of PID traces for repeatable odor delivery with a constant concentration are shown at (B): thin gray lines are individual trials, and blue line is an average profile. During the first 40 ms from the final valve trigger, odor concentration reaches 90% of its saturated value. For most of the trials, exhalation duration is longer than 40 ms. Note: conceptually the same experimental setup is used for the awake head-fixed preparation, with an addition of the water spout below the odor port (not shown). An example of the sniff rhythm in panel A is recorded in the awake state, the sniff cycle in the anesthetized state is more regular and less variable.

FIGS. 8A-8D show usage scenarios of chemical sensors using a mouse as a chemical detector with an electrode assembly.

FIGS. 9A-9B exhibit odor footprints of different odors and concentrations. Odor footprints are consistent between animals, and presents similar pattern that becomes stronger when higher concentrations are inhaled by the animal.

FIGS. 10A-10B present experimental benchmark of the bio-electronic nose vs. behaving animal. FIG. 10A shows that the bio electronic nose is as sensitive as the behaving animals, capable of detection of different odorants at very low concentrations. FIG. 10B shows the bio-electronic nose can classify different odors and even classify concentration. These parameters cannot be reported by behavioral animal training.

FIG. 11 presents a two-flap electrode designed to cover two sites on the olfactory bulb. The electrode was designed specifically for the bio-electronic nose.

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION OF VARIOUS EMBODIMENTS

Certain embodiments described herein relate generally to a mobile, highly sensitive animal-based chemical detectors that can be customized to detect a wide variety of chemicals under real-world conditions. Described herein are methods and systems for brain/machine interface devices using electrodes to read odor-signatures from the patterns of activated glomeruli, for example in the rodent olfactory bulb. This approach is referred to as a bio-electronic nose and results in a chemical sensor.

One embodiment of a chemical sensor includes an electrode assembly and a chemical detector. The electrode assembly is configured to receive a signal from the chemical detector. In one embodiment the chemical detector is an organic component, such as a cell, group of cells, cell reactor, tissue, or organism.

The electrode assembly is in communication with the chemical detector. In one embodiment, the electrode assembly includes at least one electrode in physical contact with the chemical detector. The at least one electrode may be a surface electrode, that is the electrode may be present on a surface of the chemical detector. The chemical detector transduces chemical stimuli into electrical signal that is acquired by the electrode, and is described as variations of voltage over time.

In one aspect, the chemical sensor (bio-electric nose) comprises an electrode adapted to receive signals from a cell, group of cells, cell reactor, tissue, or organism (chemical detector). For example, high density surface electrodes can be used to read and classify chemical response patterns directly from odor-evoked responses in the olfactory bulb. This approach exploits the inherent sensitivity of the olfactory system, without having to rely on noisy and unpredictable behavior as a readout. Interaction with the early olfactory processing, thus, provides an interface to the proven natural biochemical odor detectors, as opposed to signals representing ‘perceptual’ processing within the organism. The signals are indicative of the detection by the cell, group of cells, cell reactor, tissue, or organism of a chemical or aroma compound including esters, terpenes, aldehydes, ketones, amines. While prior methods relied upon LFP signal collected on the olfactory bulb, embodiments described herein collect extra-cellular signals. The proximity of each electrode to a glomerulus ensures the signal contains OSN response component. The strong coupling between the anatomy, the design of the electrodes and the data processing allows for filtration of the early olfactory response from highly correlated higher-order signals, therefore allowing for classification of odors and mixtures as well as identification of concentration, which is virtually impossible just by assessing a raw LFP electrical signal collected on the OB.

The cell, group of cells, cell reactor, tissue, or organism may be derived from or a whole organism, such as a vertebrate, more specifically a mammal, such as a rodent or canine. As an example system, mice of C57BL6/J background are utilized as a chemical sensor in communication with electrodes as a reliable, flexible and adaptable chemical sensor. A mouse senses odors with ˜5 million olfactory sensory neurons in the nasal epithelium. Each sensory neuron expresses one odorant receptor (OR) from a repertoire of over 1,000 OR genes. Olfactory sensory neurons (OSNs) that express a given OR send axonal projections to specific glomeruli in the olfactory bulb—the first brain area responsible for olfactory information processing. There are more than 2,000 individual glomeruli in the bulb (two-three glomeruli per OR in each hemisphere). Every odor triggers a unique spatiotemporal pattern of glomerular activity, which the brain decodes in order to detect and identify odorants in the olfactory world.

Further, current multi-electrode arrays can record activity primarily from the dorsal (or top) surface of the olfactory bulb. However, olfactory information is not restricted to the top of the olfactory bulb, but is directed over its entire surface, most of which is not accessible for electrode or optical recording. Thus, the sensitivity and specificity of a bio-electronic nose depends on whether glomeruli with the desired chemical specificity are located on the dorsal olfactory bulb surface. More generally, the effectiveness of a bio-electronic nose could be improved by tuning the specificity of the underlying biological olfactory system and by optimizing its anatomical organization to match the geometry of the brain machine interface.

In a further aspect, the cell, group of cells, cell reactor, tissue, or organism, are genetically modified to optimize the interaction with the electrode. Genetically modified organisms, such as mice, are adapted to allow detection of specific chemicals by targeted or modified expression of specific olfactory receptors or rearrangement of the physical location of the projections of the olfactory receptor neurons. This approach will allow us to tune the specificity and sensitivity of the olfactory receptors toward particular applications—something that is not possible in wildtype vertebrate species. For example, particular groups of cells or their projections (glomeruli) may be moved or duplicated within the larger olfactory organ for better interaction with surface electrodes.

In a further aspect, the receptor gene to be expressed may be selected or modified so as to increase its sensitivity to chemicals of interest. This modified receptor (taken from any species) would then be expressed in a genetically modified animal so that the cells or their projections are located in a position within the olfactory system so as to best interface with the electrode assembly.

Regardless of the form of the chemical detector, in one embodiment it is capable of detecting one or more chemicals in an environment. For example, the one or more chemicals may be explosives, chemicals indicative of a condition or diseased state, drugs, organics, inorganics, biological materials, etc. The chemical detector may be an olfactory receptor (OR), including as part of an olfactory sensory neuron (OSN). The OSN typically includes a dendrite and cilia that are exposed to the environment and an axon that travels to the olfactory bulb. In the olfactory bulb, multiple OSNs that have the same type of OR bundle together to form glomeruli. The glomeruli provide a point between the nose, that is the initial olfactory receptor, and the olfactory cortex where the signals are processed by the brain. Receptors of the same type project to typically two glomeruli. Thus, the glomeruli present a basic unit in mapping of chemical detection. An odor is essentially one or more chemicals being sensed by an organism. The “odorant” is sensed by the OSNs, which are selective over a range of chemicals. The combination of responses from a range of ORs provide a unique temporal and spatial response resulting in an “odor map” or “olfactory map” representative of the “odorant”. Positioning of the at least one electrode in communication with glomeruli provides for signals with minimal additional processing by the organism's brain.

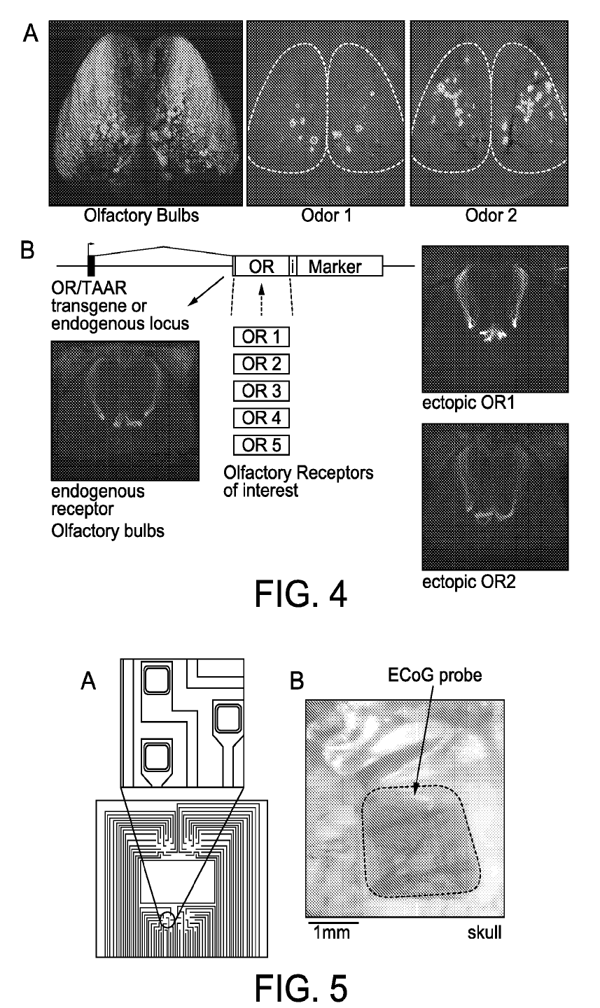

The glomerular response maps have been intensively studied using imaging methods, among which Ca2+ imaging of OSN axonal terminals is the most common (FIG. 4). The pattern of glomerular activation carries information about odor stimuli and can be used to decode odor identity and concentration. In this instance, active glomeruli are identified using imaging of transgenic mice with ORNs expressing GCaMP. Once active glomeruli per odorant are identified, those that are active in lower concentration are selected to be recorded. Using anatomical landmarks, the electrode can be then implanted covering those glomeruli. Typically, if the active site is located within the boundaries of an active glomerulus, given the size of the glomerulus (˜60-80 um) and the electrode spacing (˜50 um) proper signals can be obtained. Activation of the receptors in response to odor produces, not only the changes in Ca2+ concentration in receptor neurons, but also spiking activity of the presynaptic and postsynaptic cells and synaptic currents, which lead to changes in the local field potential (LFP) within the glomerular region. It is believed that such changes could be detected on the surface of the OB using newly developed, high-density surface electrodes. While much work has been dedicated to the study of LFP in the OB, surprisingly very little is known about the signature of the LFP on the surface of the OB.

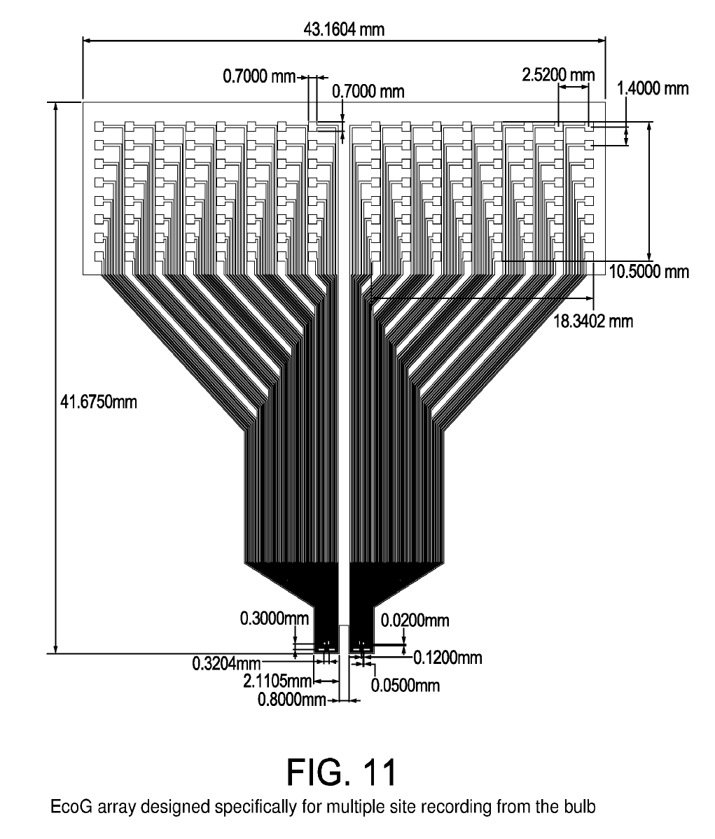

Turning back to the electrode, in one embodiment, the electrode assembly receives signals from glomeruli. Odors activate multiple glomeruli in the olfactory bulb. In one embodiment, the electrode spacing is similar to the typical size of a glomerulus. Consequently, placement of the electrode array over (such, but not limited to, spaced about 10 μm) a glomerulus ensures there will be one site receiving signals modulated by one specific glomerulus. The registration of the electrode to known glomeruli can be done by chemical calibration using ligands known to stimulate certain glomeruli or by using optical methods where certain glomeruli are marked by fluorescent markers. FIG. 11 shows a two-flap electrode designed to cover two sites on the olfactory bulb. The electrode was designed specifically for the bio-electronic nose. In cases where there is more than one site active for a given odorant or set of odorants two or more flaps of electrodes can be placed to cover two (or more) separate, distinctive sites on the olfactory bulb.

In one embodiment utilizing an organism, such as a mouse, as the chemical detector, the mouse is anesthetized and a craniotomy will be made above one of the OBs. A grid electrode will be positioned on the surface of the bulb. The recording is optimized for different conditions: sub or epi-durally or through a thin layer of skull. Alternatively, a surface electrode can be implanted in conjunction with a probe (such as silicone) inserted to the bulb to obtain signals from higher processing levels, such as the mitral/tufted cells, to improve the filtration of early olfactory responses. Typically, the electrode is divided into several areas (1-4) of 8×8 electrode sites. These grids can access different areas on the bulb. Ideally the electrode is placed over glomeruli that are active for a given set of odorants presented in low concentration.

In one embodiment, the at least one electrode is a high-density NeuroGrid (see description in Dion Khodagholy, Jennifer N Gelinas, Thomas Thesen, Werner Doyle, Orrin Devinsky, George G Malliaras and György Buzsáki NeuroGrid: recording action potentials from the surface of the brain. Nature Neuroscience, 22 Dec. 2014, incorporated herein by reference). surface electrode with an inter-electrode distance of 20-60, preferably ˜40 μm (FIG. 2). In one particular embodiment, 64, 120, 128 or 256-site arrays are used with recording site size 10 μm×10 μm, and pitch 30, 40, and 50 μm, and associated micro-connections to allow to route the recording sites to signal multiplexers. This array will cover sufficiently large portion of the dorsal bulb and allow detection of LFP signal from large numbers of glomeruli, with each single electrode with the array associated with a single glomerulus. Current technology allows coverage of most of the dorsal part of the bulb, which presents ˜50% of the glomeruli.

In one embodiment, the electrode assembly is chronically implanted in the chemical detector. High-density surface electrodes read and classify chemical neural response patterns directly from the olfactory bulb—the first part of the brain that receives chemical information. This approach exploits the inherent sensitivity of the peripheral olfactory system, without having to decode signals obtained from higher order processing of the brain, and certainly avoid relying on noisy and unpredictable behavior as a readout.

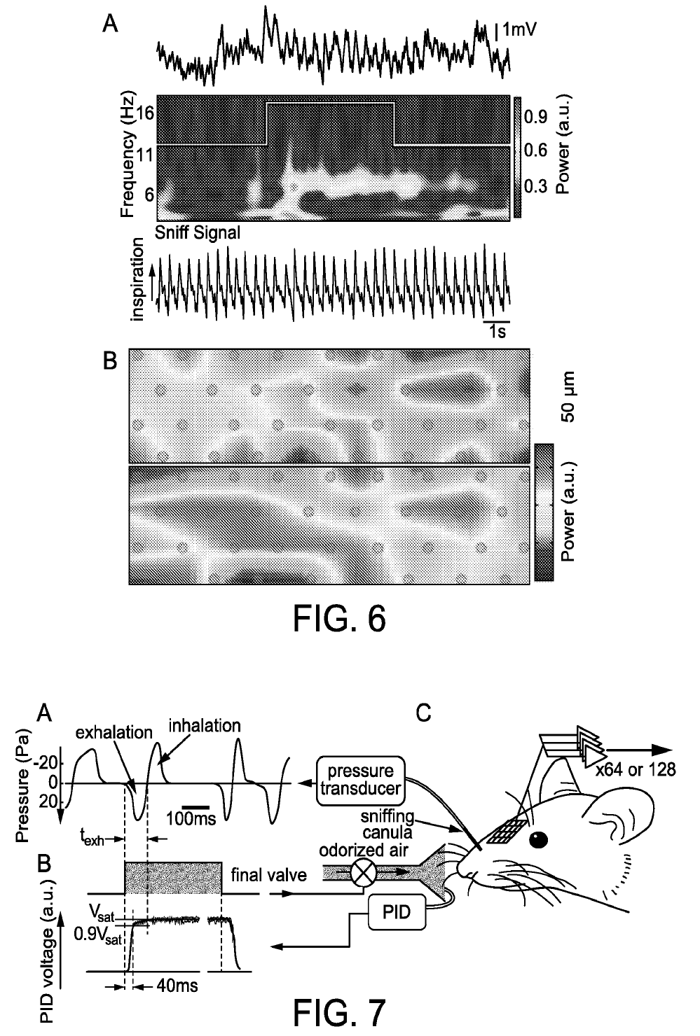

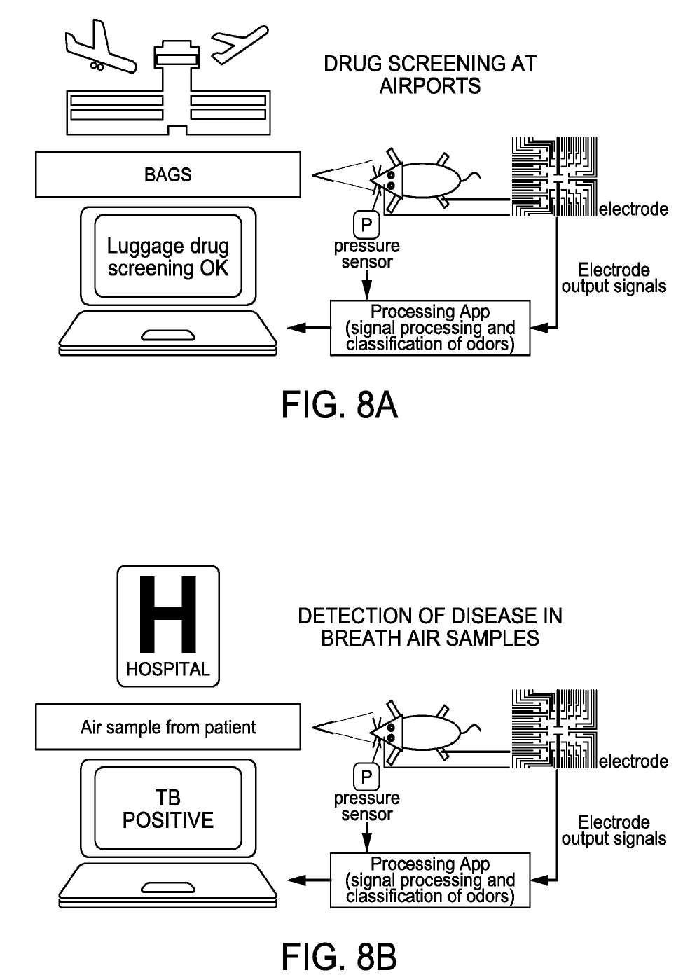

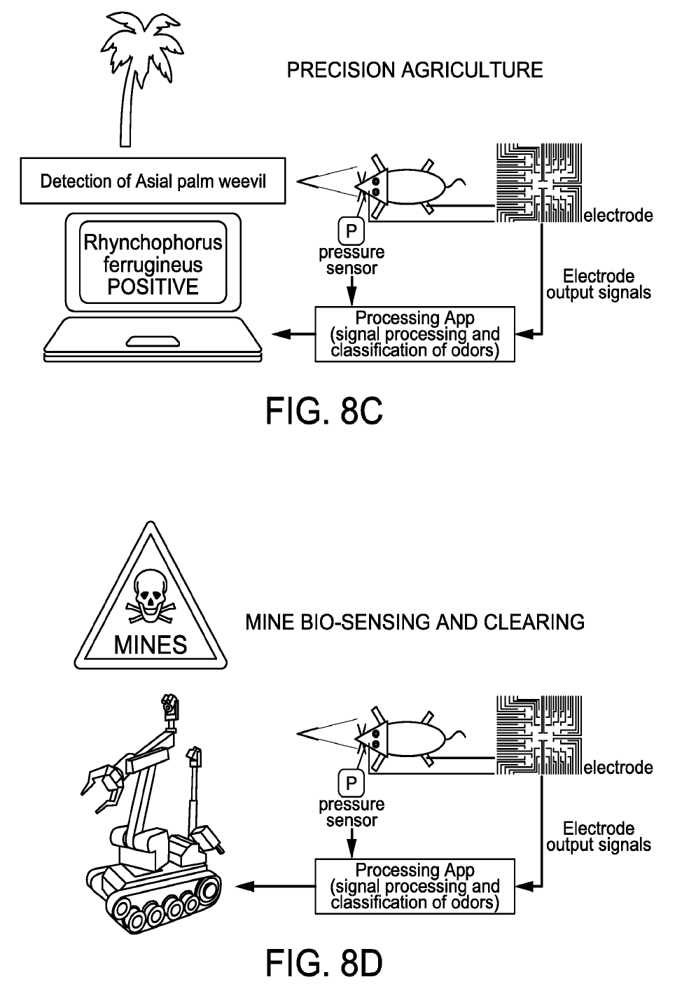

In order to extract maximum information about odor stimuli, in one embodiment, the method utilizes known libraries or undertakes sampling of the LFP with a spatial resolution that is less than typical inter-glomerular distances, i.e. 60-80 μm. In preliminary experiments, an electrode array with an inter-site distance of ˜20-50 μm (FIG. 5) was positioned on the surface of the mouse OB and recorded LFPs evoked by odors (FIG. 6). While not specifically designed for olfactory recordings, the electrode array picked up odor-specific responses locked to the sniffing cycle with high accuracy. In addition, the presence of two different odorants could be discerned by observing spatial distributions of local field potential power on the electrode array (FIG. 6).

In one embodiment, a method of interpreting information from the at least one electrode relies on the development of reliable techniques for recording on the surface of the bulb and on algorithms for extracting odor information from these recordings. To minimize development risks, we will first develop this technology in anesthetized preparations. In one embodiment, the raw signals are filtered in the frequency domain. Theta waves, indicative of respiration, are used to verify inhalation in conjunction with readings from the pressure signal. Two frequency bands are used for further processing: LFP signals (at frequencies varying between 1-100 Hz and multi-unit activity (MUA) at 300-2000 Hz). Extracted information from each electrode is re-arranged to provide temporal response vectors. These vectors are then pre-processed (by smoothing, or principle component analysis) prior to further classification processing by supervised machine learning algorithms such as linear or cubic support vector machine (SVM).

In one embodiment, a method of interpreting information from the at least one electrode relies on the development of reliable techniques for recording on the surface of the bulb and on algorithms for extracting odor information from these recordings. In one embodiment, the raw signals are fed into multi-layer convolutional neural network (CNN). The network is then trained to detect a certain target chemical in the presence of many background odors, and/or estimate the concentration of the target chemical in the given sample.

Once the CNN is trained on a large variety of different odorants with different concentrations it is able to maximize the probability of detecting a given odorant. Furthermore, if the CNN is trained with a large set of one target odorant mixed with various other odorants, it can detect the target within various different background odorants. As a useful by-product, careful definition of the first hidden layer reveals features in the input signal that characterize a certain odorant. This can be used to detect a certain odor by using very efficient, real-time processing of the electrode signal.

To develop algorithms for odor detection. The first, most basic question that we need to answer is how well different odors can be discriminated using this recording system. It is hard to estimate the number or predict the spectrum of odorants that an animal may experience in nature, so we will initially limit our stimulus set to 16-20 odorants for practical reasons. This stimulus set will contain structurally similar and dissimilar chemicals that preferentially activate the dorsal bulb, the region that is most accessible for recording. Current results show that naïve machine learning algorithms can easily classify an odor from a set of ˜10 odors, even the odorant is chemically similar to other odorants presented (as in the case of + and − carvone enantiomers).

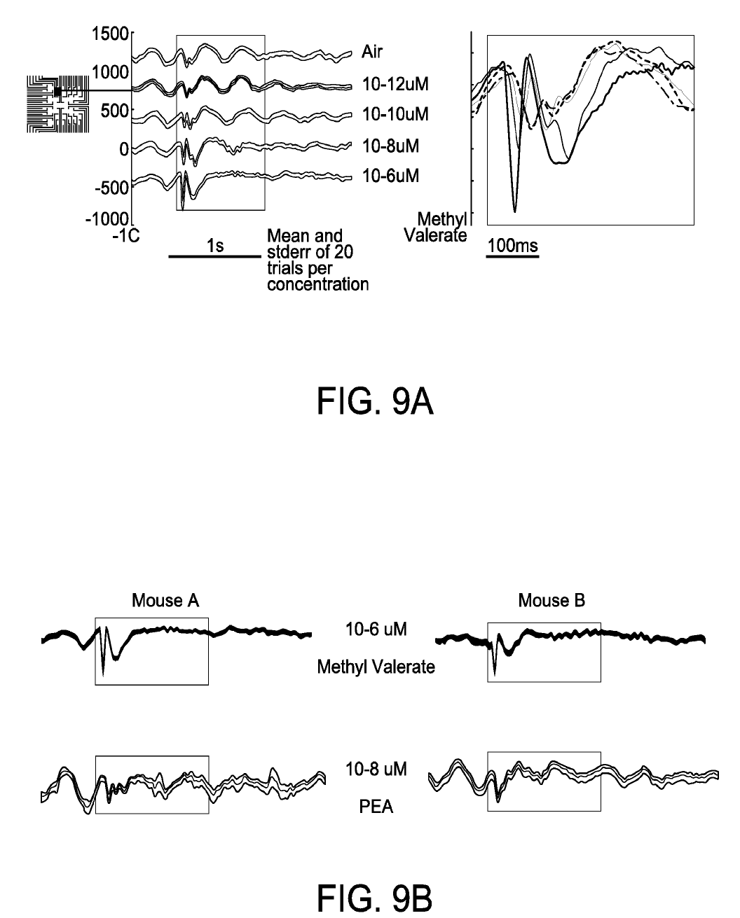

In addition to variable concentration, an odor may appear on the background of another odor. When assessing the odor detection algorithm, the criterion for successful detection would be finding a unique, odor specific fingerprint that is invariant across concentration and in the presence of additional masking odorant or odorants. In one embodiment, the chemical sensor is adapted to detect a chemical (such as an odor) even in the presence of one or more background chemicals/odors. These distinctive odor specific footprints features allow detection. A typical odor footprint is shown in FIG. 9. In some cases the distinctive parts of the odor footprint are not apparent from the temporal voltage signal obtained by the electrode. However, the classifying algorithm may identify the characteristic feature. For some classifying algorithms the characteristics features can be extracted after a CNN was trained to identify the target odorants in a large set of different odorants or in mixtures where the target odorant was present in.

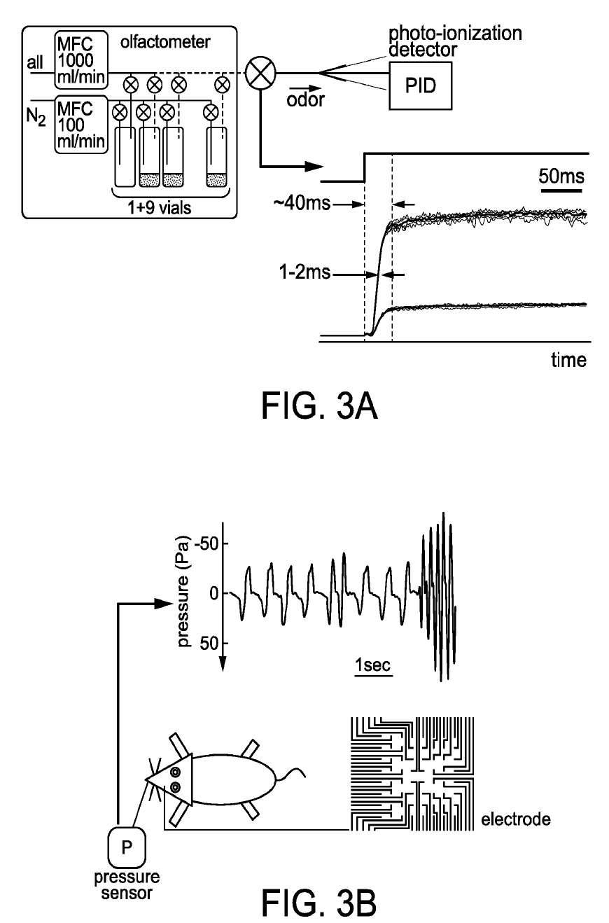

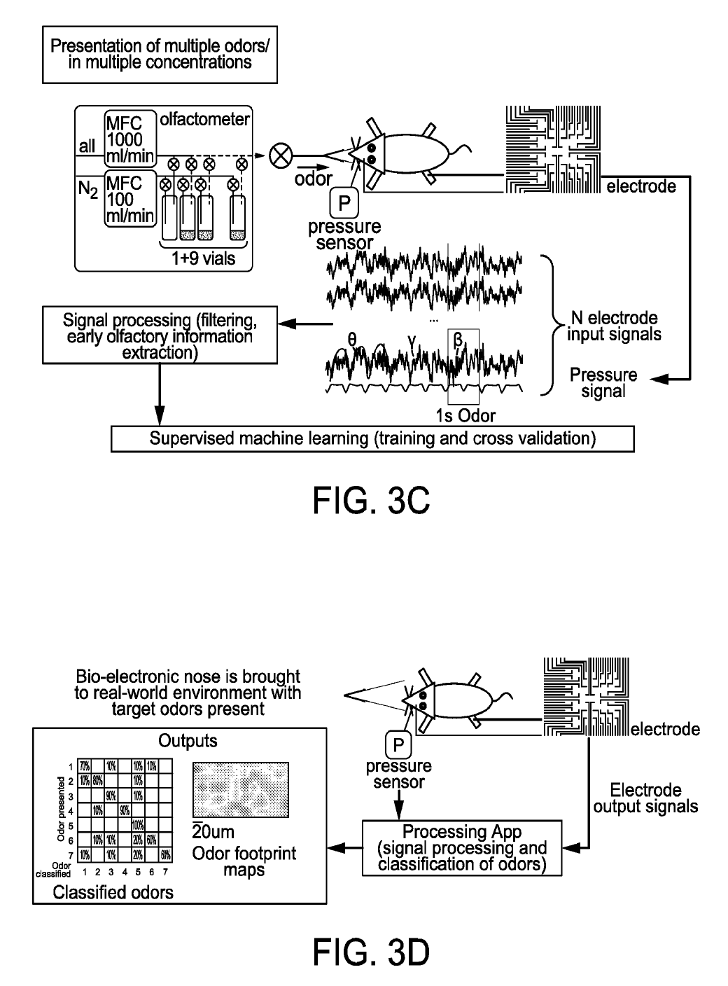

To characterize this footprint multiple strategies are deployed to decode the sensed odor from the spatial-temporal signal obtained from the OB. In one embodiment, first the temporal characteristics of LFP oscillations on individual channels are extracted (FIG. 3.), followed by extraction of the response latency from the onset of the first inhalation after the odorant was presented. Further clustering of the responses and applied machine learning techniques are planned to decode the odor given a wide dataset of different odors and concentrations.

The importance of finding a target odorant in a varied mixture of multiple different odorants plays an important role for systems and applications. For example, identification of a low concentration drug or explosive fingerprint embedded in many other odors present in the luggage of a passenger will enable detection of restricted substances in an airport. Subsequently, detection of a metabolite generated by a disease induced physiological process in a variable sample of body fluid or feces will enable applications for diagnosis of diseases.

In nature, an odor always appears at variable concentrations. One of the main challenges is to develop algorithms for concentration invariant odor identification. An increase in odor concentration leads to activation of larger numbers of glomeruli and a temporal shift of neuronal activity earlier in the sniff cycle. Using data from the recording of LFP signal in response to a few odors with multiple concentrations, allows for a technique to a) eliminate a temporal shift, and/or b) align recordings at different concentration. FIG. 9A shows that higher concentrations drive faster transient in the LFP signals. In one embodiment, the algorithm is focused on identification of the activity of smaller and unique subset of glomeruli that get activated even at the lowest presented concentration. Another approach is to find the site where the onset of the odor footprint appeared first. Transient in the odor response is indicative of activation, therefore the first site activated is very sensitive to the material even in low concentrations (FIG. 9A—onset of odor pattern happens earlier in higher concentrations).

For methods including development of an algorithm, all algorithms may be trained by a subset of data (training sub-set) and the performance will be judged using the rest of the data (testing sub-set). The stability of the analysis can be tested for the duration of the recording session, thus ensuring that a slow drift of the signal during the session won't affect the performance.

In order to train and evaluate an algorithm for detection over a background odor, the combination of different odorants, odorants in different concentrations, and combinations of two or more odorants is used to characterize the performance of the detection algorithm and allow for sensitivity analysis under varying SNR conditions and possible revision of the electrode grid's specifications. (FIG. 11)

For mice, it has been shown in previous work that the odor responses in the OB are tightly locked to the sniff cycle. For testing purposes, in order to extract odor information from LFP recordings in the most efficient manner, one method utilizes a highly precise odor delivery system with simultaneous monitoring of the sniff cycle (FIG. 7). After debugging the recording technology as necessary, odor response data is collected for different conditions, with variable levels of complexity for the decoding task. In one embodiment, odor response data recorded will include: 1) responses to a large number of odorants (16-20) with constant concentrations; 2) response to a small number of odorants (6-8) with variable concentration in the range of 6-8 orders of magnitudes; 3) responses to a few of odorants (4-6) in the presence of the background odorant(s). The recorded signals can then be used to optimize the algorithms for odor detection, by proper identification of the distinctive patterns associated with the target odorant even when presented in a mixture. Awake mice breath and sniff at a much higher frequency range (3-10 Hz), than anesthetized mice (1-2 Hz). In the awake state, some of the glomeruli follow the temporal profile of the sniffing/breathing rhythm, some do not, and some respond only intermittently to the first sniff. For embodiments using awake organisms, the algorithms should be adapted to work with signals from the more variable awake state.

As discussed above, in one embodiment the organism is awake and includes an implanted electrode. In one embodiment, the mouse will be anesthetized and implanted with a pressure cannula for recording of sniffing/breathing rhythm, a head bar for head-fixation and a grid electrode array overlying the dorsal OB. The electrode array will be implanted on the mouse olfactory bulb. In an alternative embodiment, the electrode array is connected to micro-connectors implanted on the mouse skull. The data acquisition electronics will be external to the implantation device and will be connected to the electrode array at each recording session

After recovery from the surgery, the mouse can undergo behavioral tests to obtain a reference. For example, in one testing scheme a mouse will be water deprived and acclimated to head-fixation. Water deprivation allows one to train the mouse on an odor detection paradigm, and it simplifies acclimation to being head-fixed. To calm the mouse in the head-fixation setup, occasional water rewards are provided via a waterspout positioned below the odor port. In one embodiment, the chemical sensor (rodent) requires only a short recovery (3-5 days) and a short habituation for the recording station (˜30 minutes).

For anesthetized sessions, at each session a mouse will be positioned in a head-fixed apparatus similar to that used for anesthetized recordings (FIG. 7). The sniffing/breathing pattern and signals continuously recorded from electrode array, and we will expose the mouse to multiple odor presentations (n˜500). A mouse exposed to a battery of multiple odorants, to odorants with different concentrations, and to odor mixtures of target and masking odors.

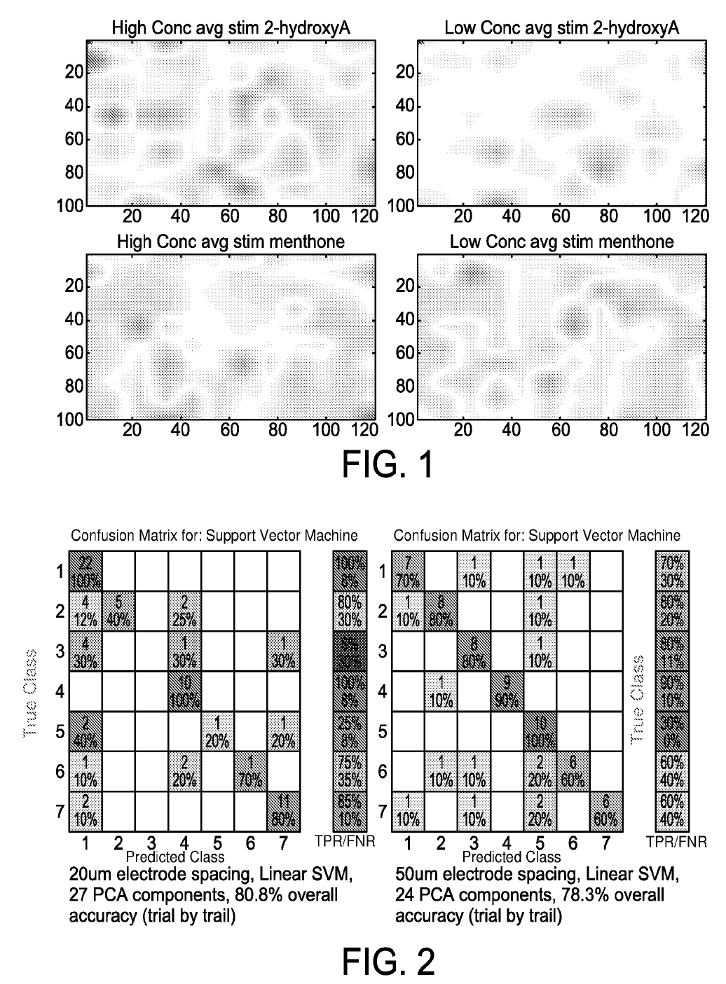

Two families of prototype electrodes featuring different coverage of the olfactory bulb and different electrode spacing were tested. After acute implantation in the anesthetized mouse we obtained signals. Processing of the signals revealed odor specific response maps, with concentration invariant signatures. This promising result (FIG. 1) indicated the concept is viable. Furthermore, we implemented machine learning algorithm to develop a classification application. The classifier yielded accuracy of ˜80% using both electrodes without extensive pre-processing of the electrodes data (FIG. 2).

A mouse with an implanted electrode array may be used in multiple sessions, which allows testing multiple different odor presentation paradigms on the same electrode array. In addition, it allows testing the stability of the recordings from session to session over long time periods.

Further, one embodiment relates to a system for measuring behavioral detection threshold in head-fixed mice. Mice implanted with a sniffing cannula and head-bar will be water deprived and acclimated to head-fixation. The training will usually start ˜2 weeks after the surgery. Mice will be first trained to lick in response to any odor presentation. A mouse will be rewarded with a drop of water for a correct lick. The odor concentration will be systematically lowered until the mice can no longer detect it. To deliver odors at various concentrations, a sequential dilution of an odorant in mineral oil or other odorless solvent. Multiple behavioral controls will be implemented to ensure that the mouse is performing the task based on odor information but not on any other cues such as valve clicks or impurities in the odor delivery system. An absolute behavioral detection threshold will be measured for a few odorants, and these odorants will be chosen to measure electrode array detection.

In addition, mice will be trained to detect a specific odor in the presence of background (masking) odors. The masking odor is delivered simultaneously with the target odor or prior to delivery of the target odor. The behavioral thresholds are tested with masking odors of different chemical similarity to the target odor.

The behavioral detection thresholds can be compared with electrode detection thresholds at least for the odorants that preferentially activate dorsal glomeruli.

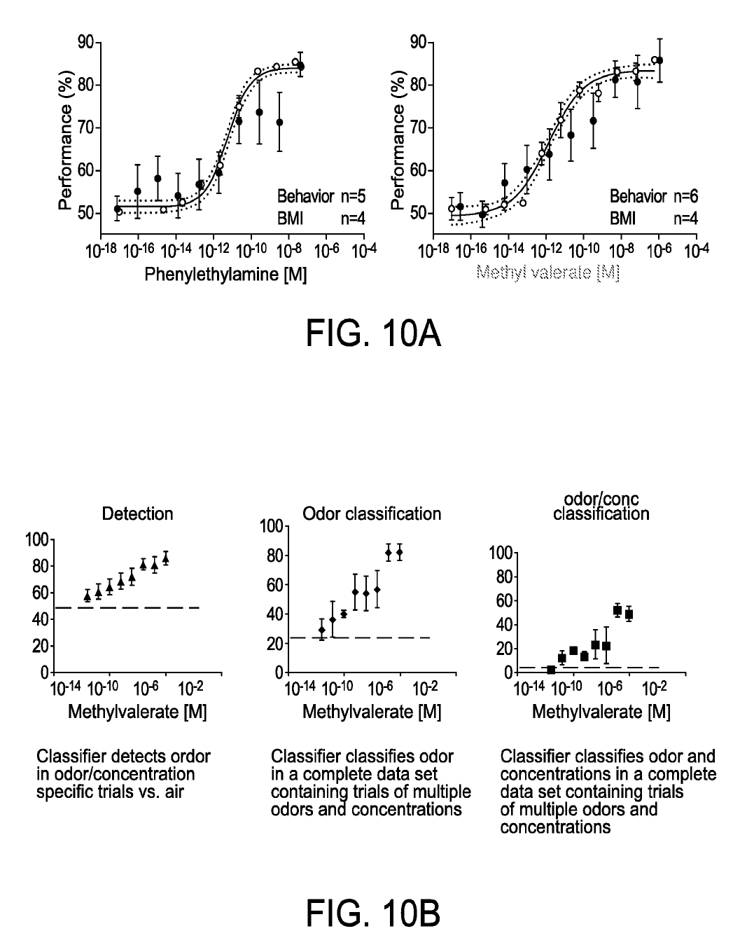

The described systems and methods enable a number of improvements in the art that were made. A bio-electronic odor detector featuring very high sensitivity (e.g. higher than electro-chemical detectors), such as greater than 4, 5, or 6 orders of magnitude of molarity. FIG. 10 shows the sensitivity of the bio-electronic nose compared to a trained animal. It is clear that the range of concentrations detected by the bio electronic nose exceeds 6 orders of magnitude of molarity, which is better than chemical detectors. In addition to obtaining comparable sensitivity to trained animals, the bio-electronic nose can also classify odorants and concentrations from the signal, making it very selective (unlike trained animals that can either detect one odorant or a family of odorants, but will not be able to report the exact odorant detected or its concentration). Applications include, but are not limited to: precision agriculture, homeland security, and disease detection.

In one embodiment, a method is provided for classification sensitivity for lower concentrations. The detection threshold is similar to behavioral training for multiple odorants, showing the electrode system is capable of reading signals that represent the odor sensed.

In one embodiment the minimum concentration (EC50) of Phenylethylamine detected by the bio-electronic nose is 10⁻¹¹M, and the minimum detectable concentration of Methyl Valerate is 10⁻¹²M (FIG. 10).

In one embodiment, systems and methods can utilize the bio invention can detect odor footprints of bacteria, tree diseases such as Rhynchophorus ferrugineus, and commercial, reliable detection of truffles.

Another embodiment relates to a bio-electronic detector for a variety of odors, including mixtures and odor footprints of odor agents representative applicative cases. It is known that mice have an exquisite sense of smell and can detect chemicals at very low concentrations. The design enables for chronic implantation of the electrode and subsequent positioning of electronics capable of recording responses, so that utilization does not require positioning of the mouse in any head fixation device. Furthermore, continuous recording of the signals and storage or wireless transmission of the data with additional meta-data such as GPS coordinate or other environmental information will unlock additional applications such as distributed mapping of complex environments using odor footprints. The grid electrode will be connected to an external multiplexing amplifier system and the LFP signal will be recorded using a 256 channel data acquisition system (OpenEphys, Intan or Neuronexus). A typical assembly of a GPS receiver, electrode, electro-physiological interface and VHF data link can be assembled on a collar/back pouch, weigh ˜50 grams and be suitable for rats.

One embodiment relates to homeland security applications, such as detection of explosives, drugs and other controlled substances.

One embodiment relates to a method for distributed odor mapping of areas. An organism, such as a rat, includes one or more electrodes mapped to glomeruli as described above. In addition, one or more of a recording system, wireless communications, GPS and a chemical sensors are provided in communication with each other and/or a central processing unit, either locally or through remote communication means. The system provides for detecting odors by freely moving organisms and recording information regarding positions of the organisms. An odor map of a target area can be created given the recorded information.

In one embodiment, the chemical sensor is affixed to, removably or fixedly, a mobile device. An organism, such as a rat, includes one or more electrodes mapped to glomeruli as described above. The mobile device may be a vehicle configured for terrestrial, aquatic, and/or atmospheric movement. A motorized vehicle, control system, odor and concentration classifier and the a chemical sensor (the organism and electrode) are in communication. The motorized vehicle is directed to determined odor sources based on algorithm processing odor and concentration gradients detected by the chemical sensor. The vehicle may be controlled, at least in part, based upon information from the chemical sensor. In one embodiment, the vehicle is directed towards increasing concentration levels of the chemical/odor. In another embodiment, the vehicle is directed to minimize the detected chemical/odor. In a specific implementations, the vehicle may be used to detect leaks along a distance, such as a pipeline, to locate a target such as an explosive in a security situation, or to find individuals in rubble following a disaster. The vehicle may also be in communication with a navigation system, such as GPS, and/or may include sensors such as optical and infrared to aid in navigating obstacles. A controls system and processor may be utilized to integrate the information from the navigation system and the chemical sensor to navigate the environment while achieve the desired goal with respect to the chemical/order (locating or avoiding).

Another embodiments relates to a method of finding one target chemical typical to an condition, such as an infectious disease (c-diff in feces samples). Early detection of disease. As non-limiting examples, the chemical sensor can be used to non-invasively detect Clostridium difficile, Colon cancer, Lung cancer, Melanoma, Tuberculosis and possibly diabetes and other diseases.

In one particular embodiment, an in-vitro assay of relevant receptors is based on the information obtained from the bio-electronic nose to further develop a chemical detector without having to use a live animal. Development of an assay may include identification of sites and relevant glomeruli to find a specific target (odorant). The receptors in the cells would be expressed. The assay would then be created by expressing the right receptors. Such an assay could then be used in the place of a live animal as part of a chemical detector.

In one particular method, one can create a library of mice (or whatever the chosen organic component is, such as other mammals or individual cells lines) that are tuned to detect a spectrum of chemicals with high sensitivity and reliability under a wide range of environmental conditions. In the future, similar manipulations might be done in any species that permits transgenesis/embryo manipulation (i.e. via CRISPR/Cas9 mediated genome editing). Further developments would include remote sensing via a fully autonomous telemetric data acquisition setup (i.e. backpack of a freely moving rat) and tuning classification algorithms for specific chemicals. In addition, we would propose to generate new mouse/rat strains that have enhanced sensitivity to specific chemicals of interest such as organic compounds used as explosives, propellants and their derivatives (e.g. 2,4- and 2,6-dinitrotoluene, pentaerythritol tetranitrate) which are currently difficult to detect using non-enhanced olfactory systems. This might be facilitated by identifying or creating OR variants (mutants) that are particularly sensitive to compounds of interest, and expressing them in rodents or other mammals. In addition, animals might also be tuned to detect the chemical signatures of nascent infections, injured humans, narcotics, and other targets in a variety of hostile environments.

In one embodiment, the chemical sensor comprises a genetically engineered mammal. Specifically, certain embodiments relate to genetic manipulations that can be used to tune the mammalian olfactory system to detect specific odorants, and to arrange olfactory inputs (glomeruli) in the dorsal olfactory bulb to optimize the extraction of information via multielectrode arrays or other brain machine interfaces.

Glomeruli are created by the axons of OSNs in the nasal cavity. There are several million OSNs in rodents, and each expresses a single OR gene, with OR genes being one of the largest gene families in many mammal species. The single expressed OR gene determines the chemical specificity of each OSN, and also directs the axon to form receptor-specific glomeruli in the olfactory bulb. In this way, all of the OSNs that express the same OR send their axons to the same glomeruli. Prior work has uncovered rules that determine the broad organization of glomeruli in the dorsal olfactory bulb. OSNs that express particular OR genes (Class I ORs, TAARs and a subset of Class II ORs) are pre-determined to project their axons to glomeruli in specific regions of the dorsal olfactory bulb that are amenable to optical and surface electrode recording. Therefore, using the promoters of these genes (or transgenes derived from these genes) enables creation of genetically modified mice that have custom glomeruli with known odor specificity located in defined regions of the dorsal olfactory bulb.

In one embodiment, the chemical detector is an organism that has been genetically engineered so that glomeruli with desired specificity are positioned or duplicated in the dorsal olfactory bulb. Where the chemical detector has been so modified, the placement and number of electrodes may be adapted to the changed position or number of glomeruli, to aid in electrophysiological detection specific chemicals using the electrode array. The genetic engineering may be used to create chemical sensors that are optimized for sensitivity or to discern between different chemicals within a related family.

Methods for rewiring the olfactory system in this way include (but are not limited to) the following:
1. Targeted expression of desired receptors via OR genes that are expressed in dorsally situated (projecting) OSNs. Specific ORs are known to be expressed selectively in OSNs that project to dorsal glomeruli. We have shown that mutating or changing the receptor in a given OR locus changes the expressed receptor but preserves the dorsal glomerular position. Using gene targeting approaches (e.g. recombination in ES cells or CRISPR/Cas9 methods), we can direct expression of specific receptors from model OR loci (such as taar4, olfr545, or olfr160) which results in generation of dorsal glomeruli with known chemical specificity. In this way, desired ORs can be expressed using these endogenous OR gene loci that will result in formation of dorsal glomeruli with defined chemical specificity.
2. Transgenic expression of desired receptors in dorsally projecting OSNs. Class I ORs and TAAR genes are expressed primarily in OSNs that project to the dorsal olfactory bulb. Both sets of genes are located in large genomic clusters (on mouse chromosome 7 for Class I ORs and on mouse chromosome 10 for TAARs). We have discovered cis acting enhancers that are necessary for expression of these genes in dorsally projecting OSNs. We have used these genetic elements in transgenes to drive expression in dorsal OSNs. Using this approach, desired ORs can be expressed using these genetic elements that will result in formation of dorsal glomeruli with defined chemical specificity.
3. Duplication of glomeruli corresponding to specific TAAR or OR glomeruli. Inclusion of one or more identified cis acting enhancers in OR/TAAR transgenes, or presence of multiple transgene inserts per genome (as part of normal pronuclear transgenesis) can increase the number of OSNs that express a given receptor, and the number of glomeruli formed by their axons. We exploit this finding to generate mice with an overrepresentation of OSNs and dorsal glomeruli corresponding to a desired receptor. In this way, desired glomeruli can be overrepresented by the formation of multiple dorsal glomeruli with defined chemical specificity to provide multiple iterated readouts for the electrode array.
4. Directed expression of clustered OR genes in dorsally projecting OSNs using OR enhancers. A majority of OR genes are found in large genomic clusters. The expression of these genes is influenced by local, cis acting enhancers. We have identified cis acting enhancers in TAAR and Class I OR gene clusters that drive expression in dorsally projecting OSNs. Using gene targeting approaches (e.g. recombination in ES cells or CRISPR/Cas9 methods), we can insert “dorsal” enhancers ectopically into OR gene clusters to misdirect expression of these genes in dorsally projecting OSNs. Using this approach, expression of clustered ORs with desired specificity can be shifted to dorsally projecting OSNs, resulting in the formation of multiple dorsal glomeruli with defined chemical specificity.

As used herein, the singular forms “a”, “an” and “the” include plural referents unless the context clearly dictates otherwise. Thus, for example, the term “a member” is intended to mean a single member or a combination of members, “a material” is intended to mean one or more materials, or a combination thereof. As used herein, the terms “about” and “approximately” generally mean plus or minus 10% of the stated value. For example, about 0.5 would include 0.45 and 0.55, about 10 would include 9 to 11, about 1000 would include 900 to 1100.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Other substitutions, modifications, changes and omissions may also be made in the design, operating conditions and arrangement of the various exemplary embodiments without departing from the scope of the present invention.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

1.-12. (canceled)

13. A chemical sensor comprising:
an electrode assembly; and
a chemical detector comprising an olfactory system in communication with the electrode assembly.

14. The chemical sensor of claim 13, wherein an electrode of the electrode assembly is positioned on an olfactory system within 10 μm of a glomerulus of the olfactory system.

15. The chemical sensor of claim 14, wherein the electrode assembly comprises a plurality of electrodes and the chemical detector comprises a plurality of glomeruli, each electrode of the plurality of electrodes positioned within 10 μm of an associated glomerulus.

16. The chemical sensor of claim 15, wherein the plurality of electrodes are spaced 20-50 μm apart.

17. The chemical sensor of claim 13, wherein the olfactory system comprises a glomeruli of a normal or genetically manipulated non-human mammal.

18. A system for driving a motorized vehicle based on odor detection comprising the chemical sensor of claim 13, further comprising a motorized vehicle, control system, odor and concentration classifier.

19. A method of detecting the concentration of a present chemical comprising:
binding a chemical to an olfactory receptor in an olfactory sensory neuron of an olfactory system;
detecting an electrochemical change in the olfactory system; and
identifying the concentration of the chemical based upon the detected electrochemical change.

20. The method of claim 19, wherein the identifying further comprises identifying the compounds of the chemical based upon the detected electrochemical change.

21. The method of claim 20, further comprising an electrode assembly on the olfactory

22. A method of driving a motorized vehicle based on the identification of concentration of claim 19, further comprising compiling an odor map of a target area.

23. The method of claim 19, wherein detecting an electrochemical change comprises detecting via an electrode of an electrode assembly is positioned on the olfactory system within 10 μm of a glomerulus of the olfactory system.

24. The method of claim 23, wherein the electrode assembly comprises a plurality of electrodes and the chemical detector comprises a plurality of glomeruli, each electrode of the plurality of electrodes positioned within 10 μm of an associated glomerulus.

25. The method of claim 24, wherein the plurality of electrodes are spaced 20-50 μm apart.

26. The method of claim 19, wherein the olfactory system comprises a glomeruli of a normal or genetically manipulated non-human mammal.

27. A method for distributed odor mapping of areas comprising:
providing a recording system, wireless communications, GPS and a chemical sensor in communication with each other;
detecting odors by freely moving organisms and recording information regarding positions of the organisms; and
compiling an odor map of a target area given the recorded information.

28. The method of claim 27, wherein the freely moving organisms comprise transgenic non-human mammal having a gene sequence encoding for the mis-expression or overexpression of one or more olfactory receptor genes.
